(19) United States
(12) Patent Application Publication
(10) Pub. No.: US 2026/0096887 A1
(43) Pub. Date: Apr. 9, 2026

(54) ADJUSTABLE ENDOBRONCHIAL VALVE

(71) Applicant: GYRUS ACMI, INC. D.B.A OLYMPUS SURGICAL TECHNOLOGIES AMERICA, Westborough, MA (US)

(72) Inventor: Joey Magno, Dudley, MA (US)

(21) Appl. No.: 19/346,065

(22) Filed: Sep. 30, 2025

Related U.S. Application Data
(60) Provisional application No. 63/704,302, filed on Oct. 7, 2024.

(51) Int. Cl. A61F 2/24; A61F 2/04

(52) U.S. Cl. CPC A61F 2/2476; A61F 2002/043; A61F 2250/001

(57) ABSTRACT

A bronchial flow control device comprises a tubular body comprising struts extending between first and second ends, the struts being expandable between radially contracted and expanded shapes, a valve disposed proximate the second end, a membrane covering the struts, and microfibers on the membrane. A method comprises attaching a bronchial valve to a delivery catheter in an unexpanded state, inserting the bronchial valve into an airway, actuating the delivery catheter to radially expand the bronchial valve, pushing microfibers into an anatomic duct wall, detaching the delivery catheter from the bronchial valve, and withdrawing the delivery catheter from the airway. A delivery system for a valve comprises a handle, a first actuator to control a rotational position of a first coupler, a second actuator to control a radial position of a second coupler, and a third actuator to control an axial position between the first and second couplers.

### PRIORITY CLAIM

This application claims the benefit of priority to U.S. Provisional Patent Application Ser. No. 63/704,302, filed Oct. 7, 2024, the contents of which are incorporated herein by reference.

### TECHNICAL FIELD

The present disclosure is generally directed to devices, systems and methods for treating Chronic Obstructive Pulmonary Disease (COPD) and other conditions. More specifically, the present disclosure is directed to providing an anchored intra-bronchial obstruction that may be removed.

### BACKGROUND

Chronic Obstructive Pulmonary Disease (COPD) has become a major cause of morbidity and mortality in the United States over the last three decades. COPD is characterized by the presence of airflow obstruction due to chronic bronchitis or emphysema. The airflow obstruction in COPD is due largely to structural abnormalities in the smaller airways. Important causes are inflammation, fibrosis, goblet cell metaplasia, and smooth muscle hypertrophy in terminal bronchioles.

The incidence, prevalence, and health-related costs of COPD are on the rise. Mortality due to COPD is also on the rise. In 1991, COPD was the fourth leading cause of death in the United States and had increased 33% since 1979. COPD affects the patient's whole life. It has three main symptoms: cough; breathlessness; and wheeze. At first, breathlessness may be noticed when running for a bus, digging in the garden, or walking uphill. Later, it may be noticed when simply walking in the kitchen. Over time, it may occur with less and less effort until it is present all of the time. COPD is a progressive disease and currently has no cure. Current treatments for COPD include the prevention of further respiratory damage, pharmacotherapy, and surgery. Each is discussed below.

The prevention of further respiratory damage entails the adoption of a healthy lifestyle. Smoking cessation is believed to be the single most important therapeutic intervention. However, regular exercise and weight control are also important. Patients whose symptoms restrict their daily activities or who otherwise have an impaired quality of life may require a pulmonary rehabilitation program including ventilatory muscle training and breathing retraining. Long-term oxygen therapy may also become necessary.

Pharmacotherapy may include bronchodilator therapy to open up the airways as much as possible or inhaled beta-agonists. For those patients who respond poorly to the foregoing or who have persistent symptoms, ipratropium bromide may be indicated. Further, courses of steroids, such as corticosteroids, may be required. Lastly, antibiotics may be required to prevent infections and influenza and pneumococcal vaccines may be routinely administered. Unfortunately, there is no evidence that early, regular use of pharmacotherapy will alter the progression of COPD.

About 40 years ago, it was first postulated that the tethering force that tends to keep the intrathoracic airways open was lost in emphysema and that by surgically removing the most affected parts of the lungs, the force could be partially restored. Although the surgery was deemed promising, the lung volume reduction surgery (LVRS) procedure was abandoned. LVRS was later revived. In the early 1990's, hundreds of patients underwent the procedure. However, the procedure fell out of favor when Medicare stopped reimbursing for LVRS. Unfortunately, data is relatively scarce and many factors conspire to make what data exists difficult to interpret. The procedure is currently under review in a controlled clinical trial. However, what data does exist tends to indicate that patients benefited from the procedure in terms of an increase in forced expiratory volume, a decrease in total lung capacity, and a significant improvement in lung function, dyspnea, and quality of life. Improvements in pulmonary function after LVRS have been attributed to at least four possible mechanisms.

These include enhanced elastic recoil, correction of ventilation/perfusion mismatch, improved efficiency of respiratory musculature, and improved right ventricular filling.

Lastly, lung transplantation is also an option. Today, COPD is the most common diagnosis for which lung transplantation is considered. Unfortunately, this consideration is given for only those with advanced COPD. Given the limited availability of donor organs, lung transplant is far from being available to all patients.

There is a need for additional non-surgical options for permanently treating COPD. Currently, there are non-surgical apparatuses and procedures for lung volume reduction by obstructing the air passageway that communicates with the portion of the lung to be collapsed.

The therapy includes placing an obstruction in the air passageway that prevents inhaled air from flowing into the portion of the lung to be collapsed, while permitting air and mucus from the portion of the lung to escape past the obstruction. Lung volume reduction with concomitant improved pulmonary function may be obtained without the need for surgery. The effectiveness of obstructions may be enhanced if it is anchored in place. The effectiveness may also be enhanced if the obstruction is removable.

In view of the foregoing, there is a need in the art for a new and improved device, system and method for permanently or reversibly obstructing an air passageway that is anchored in place, and that may be removed if required or desired.

### SUMMARY

The present disclosure recognizes that problems to be solved with conventional COPD devices include, among other things, 1) difficulty in loading the devices to a delivery system due to the need, for example, to hold the device in a radially collapsed state, 2) difficulty in expanding the devices to a proper size to engage anatomic ducts (e.g., lumen, airways, etc.) of differing cross-sectional sizes and shapes due to the devices typically being configured to expand to a fixed size, 3) the inability to completely or nearly completely stop inflow while also permitting outflow due to some valves completely obstructing flow, 4) difficulty in preventing migration of the device after deployment without inducing localized tissue trauma due to the use of attachment barbs that pierce tissue to anchor the device, and 5) difficulty in recovering the devices after deployment due to the need to separate the attachment barbs from tissue and collapse and hold the device in a collapsed state for withdrawal from the anatomy. The present disclosure can help provide solutions to these and other problems by providing devices, systems and methods relating to 1) devices that can easily and quickly be loaded to a delivery system such as by the device being able to exist in a collapsed state without an external constraint, 2) devices that can be expanded to a variety of different sizes such as by the device being intraoperatively adjusted to an infinitesimal number of positions by mechanical deformation, 3) the use of a one-way valve that can block flow in one direction to prevent inhalation and permit flow in the opposite direction to allow leaked air and fluid to escape, 4) the use of microfibers that can attach the devices to anatomic surfaces to prevent migration without causing localized tissue trauma, and 5) devices that can be reattached to a delivery system within the anatomy to return the devices to a collapsed state without causing localized tissue trauma.

The present invention is directed toward improved devices, systems and method for treating COPD and other conditions. The present disclosure relates generally to valves that have a pre-contracted state so that the valve can be deployed and expanded to a custom size to seal an airway. The pre-contracted state can allow the device to be loaded to a delivery system without difficulty. More particularly, the present application relates to valves having membranes that can seal a frame of the valve to prevent fluid flow through walls of the valve. The membranes can include microfibers, such as mushroom head shaped microfibers, that can adhere to tissue without cause trauma.

In an example, a bronchial flow control device can comprise a tubular body comprising a first end, a second end and a plurality of struts extending between the first end and the second end, wherein the plurality of struts is configured to expand from a radially contracted shape to a radially expanded shape, a valve disposed proximate the second end, a membrane covering the plurality of struts, and a plurality of microfibers, disposed about an exterior of the membrane.

In another example, a method of deploying a bronchial valve in an airway can comprise attaching the bronchial valve to a delivery catheter in an unexpanded state, inserting the delivery catheter with the bronchial valve into the airway, operating an actuator of the delivery catheter to radially expand the bronchial valve, pushing microfibers of the bronchial valve into an anatomic duct wall of the airway, detaching the delivery catheter from the bronchial valve, and withdrawing the bronchial valve from the airway.

In an additional example, a delivery system for a valve can comprise a handle, a first actuator connected to the handle to control a rotation position of a first coupler, a second actuator connected to the handle to control a radial position of a second coupler, and a third actuator to control an axial position between the first coupler and the second coupler.

### BRIEF DESCRIPTION OF DRAWINGS

FIG. 1 is a perspective view of a proximal end of a bronchial valve of the present application showing a coupler, a membrane and a valve assembly.

FIG. 2 is a perspective view of a distal end of the bronchial valve of FIG. 1 showing the valve assembly, the membrane and the coupler.

FIG. 3 is an exploded view of the bronchial valve of FIG. 2 showing a valve housing, a valve member, a valve frame and the membrane.

FIG. 4 is a side view of the bronchial valve of FIG. 1 through FIG. 3 showing a profile of the valve.

FIG. 5 is a cross-sectional view of the bronchial valve of FIG. 4 taken along section 5-5 showing coupling of the valve member to the frame by the valve housing and the membrane positioned around the frame.

FIG. 6 is a perspective cross-sectional view of the bronchial valve of FIG. 4 taken along section 6-6 showing struts of the valve frame.

FIG. 7 is a perspective view of a proximal end of the bronchial valve frame of FIG. 3 and FIG. 5 showing a plurality of struts connecting proximal and distal end rings.

FIG. 8 is a perspective view of a distal end of the bronchial valve frame of FIG. 3 and FIG. 5 showing the plurality of struts connecting the proximal and distal end rings.

FIG. 9A is a cross-sectional view of a bronchial valve of the present disclosure including axial struts in an elongate, undeployed state.

FIG. 9B is a cross sectional view of the bronchial valve of FIG. 9A in an axially contracted and radially expanded, deployed state.

FIG. 10A is a cross-sectional view of a bronchial valve of the present disclosure including spiral struts in an elongate, undeployed state.

FIG. 10B is a cross sectional view of the bronchial valve of FIG. 10A in an axially contracted and radially expanded, deployed state.

FIG. 11A is a schematic close-up view of a membrane of the present disclosure having an outer surface with microfibers positioned proximate to a surface to which the microfibers are to be adhered.

FIG. 11B is a schematic close-up view of the membrane of FIG. 11A being brought into engagement with the surface.

FIG. 11C is a schematic close-up view of the membrane of FIG. 11B showing the microfibers stretching as the surface is displaced.

FIG. 12A is a cross-sectional view of a handle of an insertion catheter for the valve of the present disclosure comprising a locking knob, deployment knob and an attachment knob to deploy, expand and release the valves.

FIG. 12B is cross-sectional view of the handle of FIG. 12A with the locking knob in a retracted state to pull an actuation cable.

FIG. 12C is a cross-sectional view of the handle of FIG. 12B with the deployment knob actuated to retract the locking knob and the actuation cable.

FIG. 13 is a partial cross-sectional view of a distal attachment and deployment mechanism of the insertion catheter of FIG. 12A through FIG. 12C.

FIG. 14 is a schematic cross-sectional view of the distal attachment and deployment mechanism of FIG. 13 showing a spring-loaded moving block.

FIG. 15 is a side cross-sectional view of a bronchial valve of the present disclosure with the distal attachment and deployment mechanism of FIG. 14 inserted therein.

FIG. 16 is a cross-sectional view of a bronchial valve of the present disclosure loaded onto an insertion catheter and inserted into an anatomic passage.

FIG. 17 is a schematic side view of a bronchial valve of the present disclosure deployed within an anatomic passage in an over-expanded state.

FIG. 18 is a schematic side view of a bronchial valve of the present disclosure deployed within an anatomic passage in an under-expanded state.

FIG. 19 is a cross-sectional view of a flapper valve suitable for use with the valves of the present disclosure.

FIG. 20 is a block diagram illustrating methods of deploying and recovering bronchial valves of the present disclosure.

### DETAILED DESCRIPTION

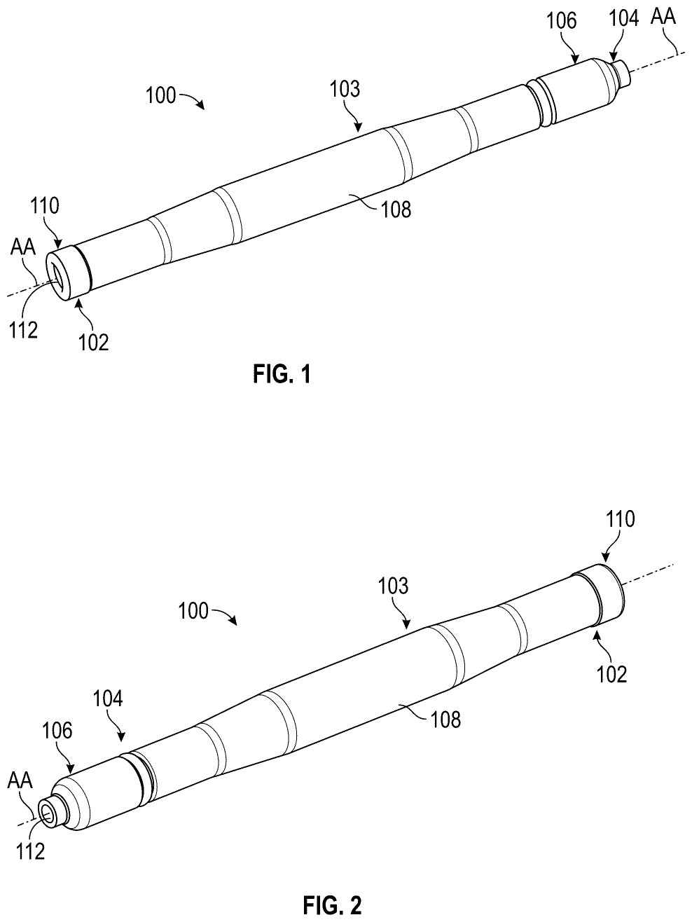

FIG. 1 and FIG. 2 are perspective views of bronchial valve 100 extending between proximal end 102 and distal end 104. FIG. 1 is a perspective view of proximal end 102 of bronchial valve 100. FIG. 2 is a perspective view of distal end 104 of bronchial valve 100.

Bronchial valve 100 can include central region 103 between proximal end 102 and distal end 104. Bronchial valve 100 can comprise proximal end 102, central region 103, distal end 104, valve assembly 106, membrane 108 and coupler 110. FIG. 1 and FIG. 2 are discussed concurrently.

Bronchial valve 100 can extend along central axis AA from proximal end 102 to distal end 104. Bronchial valve 100 can comprise an annular device having lumen 112 extending therethrough from proximal end 102 to distal end 104. Bronchial valve 100 can be configured for deployment in an anatomic duct or passage formed of tissue or biological matter. In examples, bronchial valve 100 can be positioned within passages of a lung, such as bronchi and bronchioles, to control flow of air and liquids, therethrough. Although described with reference to being deployed within a lung, bronchial valve 100 can be used or configured for use in other anatomic regions, such as by incorporating other types of valves including two-way valves.

FIG. 1 and FIG. 2 illustrate bronchial valve 100 in a collapsed state ready to be navigated through and inserted into anatomy. As discussed herein, central region 103 of bronchial valve 100 can be expanded to engage walls of the passageway when deployed.

Valve assembly 106 can be located at distal end 104 to selectively obstruct lumen 112. As described below, valve assembly 106 can comprise a valve configured to allow fluid flow in one direction through lumen 112 and prevent or inhibit fluid flow in the opposite direction through lumen 112. In examples, fluids can comprise air and biological fluids such as mucus.

Valve assembly 106 is shown and described as being located at distal end 104, but can be located in other positions, such as immediately downstream of coupler 110 or other locations.

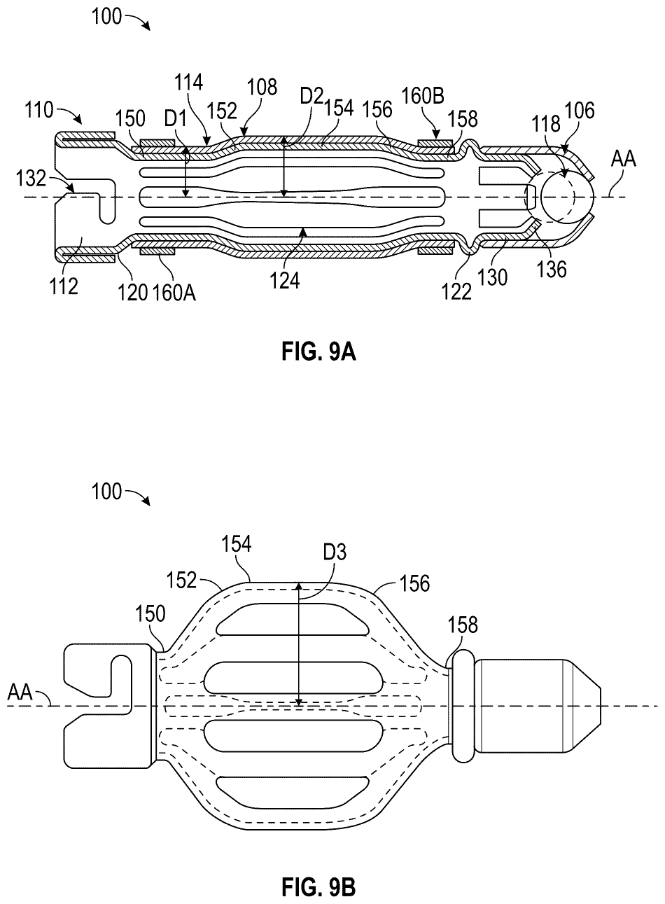

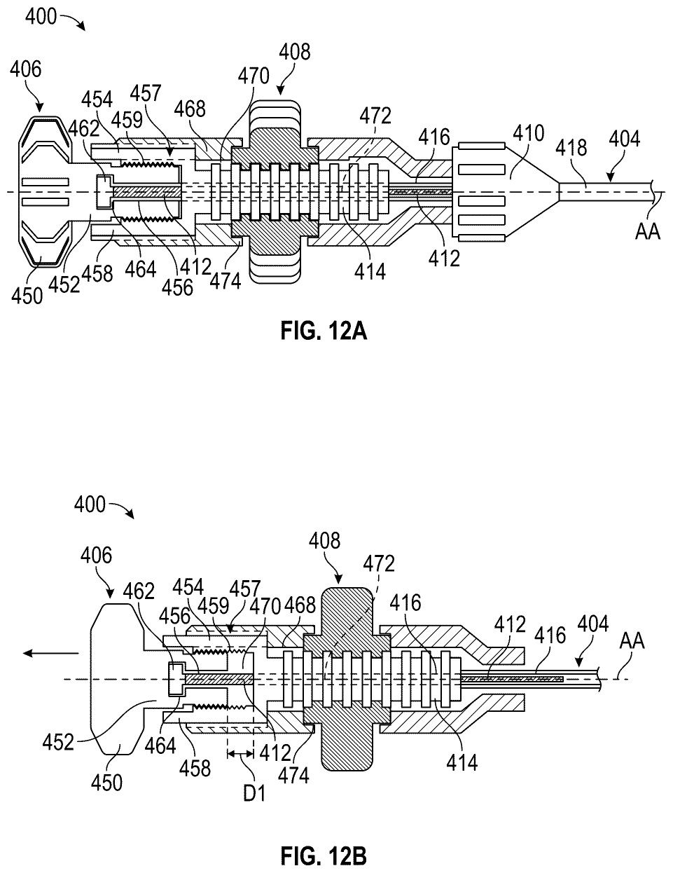

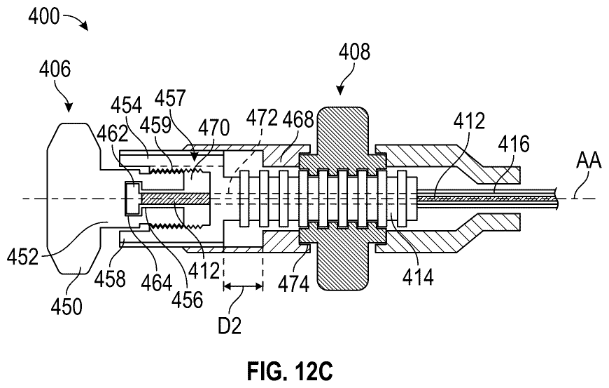

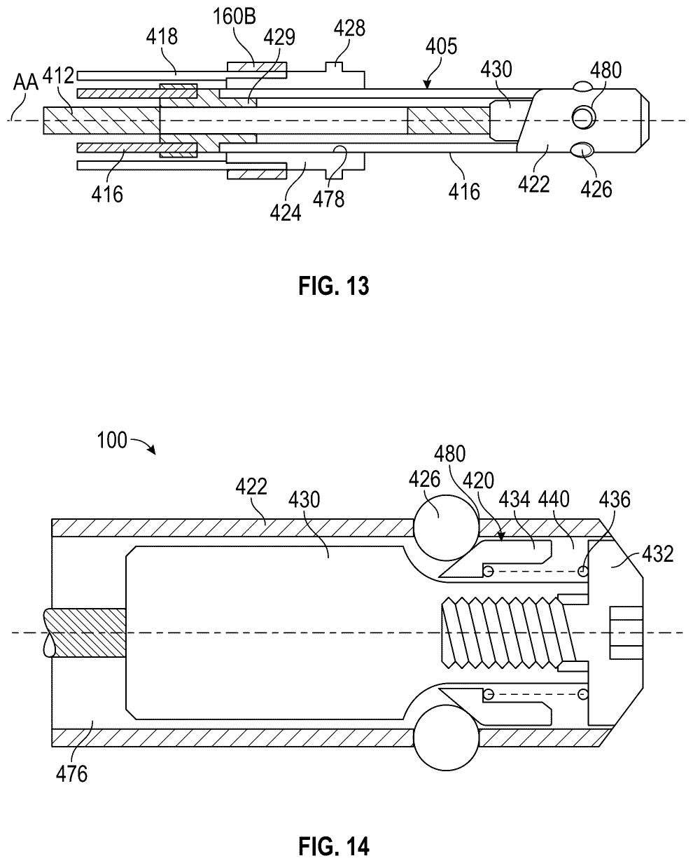

Coupler 110 can be configured to attach to a delivery system, such as delivery system 400 of FIG. 12A and FIG. 13. As discussed below with reference to FIG. 9A and FIG. 14, for example, coupler 110 can comprise a twist lock coupling that allows for a fixed coupling to the delivery system. However, other types of couplers can be used.

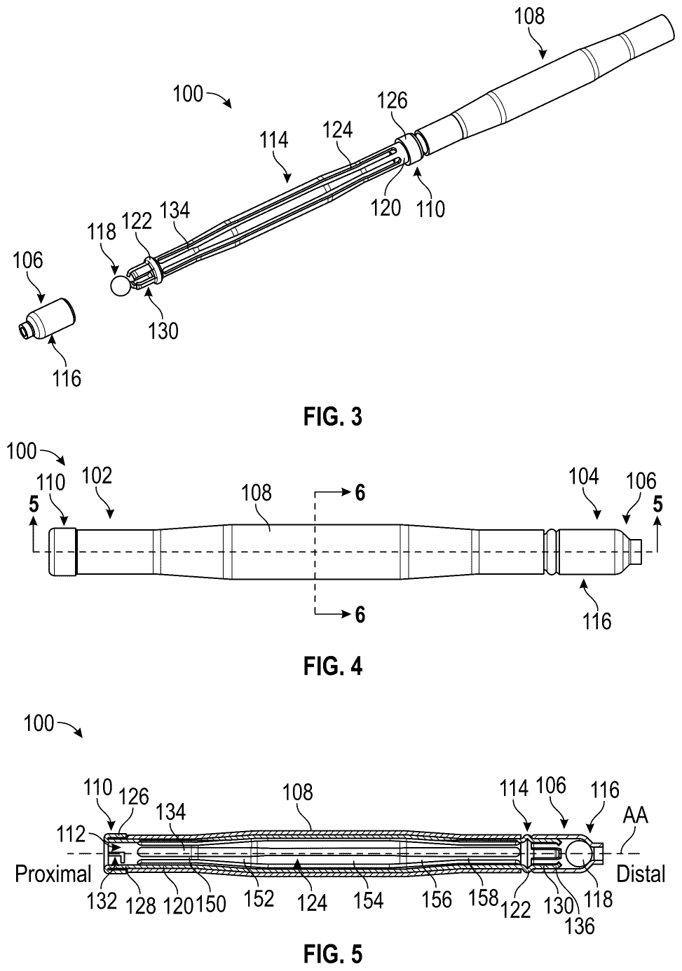

Membrane 108 can extend between coupler 110 and valve assembly 106. Membrane 108 can be disposed over valve frame 114 (FIG. 3) connecting valve assembly 106 and coupler 110. Membrane 108 can form a seal against tissue to which it engages. Furthermore, as discussed in detail herein, membrane 108 can stretch to allow for deployment, e.g., radial expansion, of bronchial valve 100.

Bronchial valve 100 can be implanted into biological anatomy to control flow of fluid through a passageway. In particular, bronchial valve 100 can be positioned and oriented to prevent air from being introduced into a non-functioning lobe of a lung during inhalation, but to allow any air or fluid to flow out during exhalation. As is understood in the art, it can be advantageous to occlude non-functioning lobes to allow other functioning lobes to utilize more of the inhaled air and to grow. Additionally, valves of the present disclosure can allow air and liquid within non-functioning lobes, which may arise due to cross-leaking of air between lobes of the lung, to escape, which can relieve pressure on the diaphragm and improve breathing.

FIG. 3 is an exploded view of bronchial valve 100 of FIG. 2 showing valve assembly 106, membrane 108, coupler 110 and valve frame 114. FIG. 4 is a side view of bronchial valve 100 of FIG. 1 through FIG. 3 showing a profile of bronchial valve 100. FIG. 5 is a cross-sectional view of bronchial valve 100 of FIG. 4 taken along section 5-5. FIG. 3 through FIG. 5 are discussed concurrently.

Valve assembly 106 can comprise valve housing 116 and ball 118. Valve frame 114 can comprise proximal end ring 120, distal end ring 122 and struts 124 extending therebetween. Coupler 110 can extend from proximal end ring 120 and can comprise outer ring 126 and inner ring 128 (FIG. 5). Valve frame 114 can further comprise fingers 130 that can extend from distal end ring 122 to engage with ball 118.

As can be seen in FIG. 5, coupler 110 can comprise a double-layered portion of material. In examples, coupler 110 can comprise a folded over portion of material of valve frame 114. Coupler 110 can comprise a stiffened portion of bronchial valve 100 that can facilitate connection to delivery system 400 of FIG. 12A and FIG. 13. Outer ring 126 can provide stiffness to inner ring 128. Outer ring 126 can additionally form a ridge that provides a barrier for membrane 108. Inner ring 128 can include keyway 132 for connection to locking pegs 428 (FIG. 13) of delivery system 400. In example, coupler 110 can include four of keyway 132 spaced approximately ninety-degrees apart.

Proximal end ring 120 can connect to coupler 110 and can comprise a portion of the material of valve frame 114. In particular, proximal end ring 120 can extend from inner ring 128. Struts 124 can extend distally from inner ring 128. Proximal end ring 120 and distal end ring 122 are shown having similar diameters. Having equal diameters that are both small can facilitate the use of delivery catheters that are also small. However, in other examples, proximal end ring 120 and distal end ring 122 can have different diameters.

Distal end ring 122 can comprise a portion of the material of valve frame 114. In examples, distal end ring 122 can comprise a corrugated portion of the material of valve frame 114 that provide stiffness and rigidity to valve frame 114. Additionally, corrugation of distal end ring 122 can form a ridge that provides a barrier for membrane 108. Struts 124 can extend proximally from distal end ring 122. In examples, distal end ring 122 can be formed using compression and rotary beading of a sheet metal tube.

In examples, valve frame 114 can be formed of a stainless-steel tube that is laser cut to form struts 124, fingers 130 and keyway 132. Further machining and processing can be used to form outer ring 126 and distal end ring 122. In examples, valve frame 114 can be formed of regular SS-304 hypo tube.

Membrane 108 can surround valve frame 114 to contact the radially outer surfaces of struts 124. Membrane 108 can cover slots 134 located between struts 124. Membrane 108 can extend between outer ring 126 and distal end ring 122. Membrane 108 can comprise a waterproof or impermeable material that can seal valve frame 114 against tissue. Specifically, membrane 108 can prevent fluid within valve frame 114 from escaping radially outward from within valve frame 114, thereby forcing the fluid to have to flow in an axial direction.

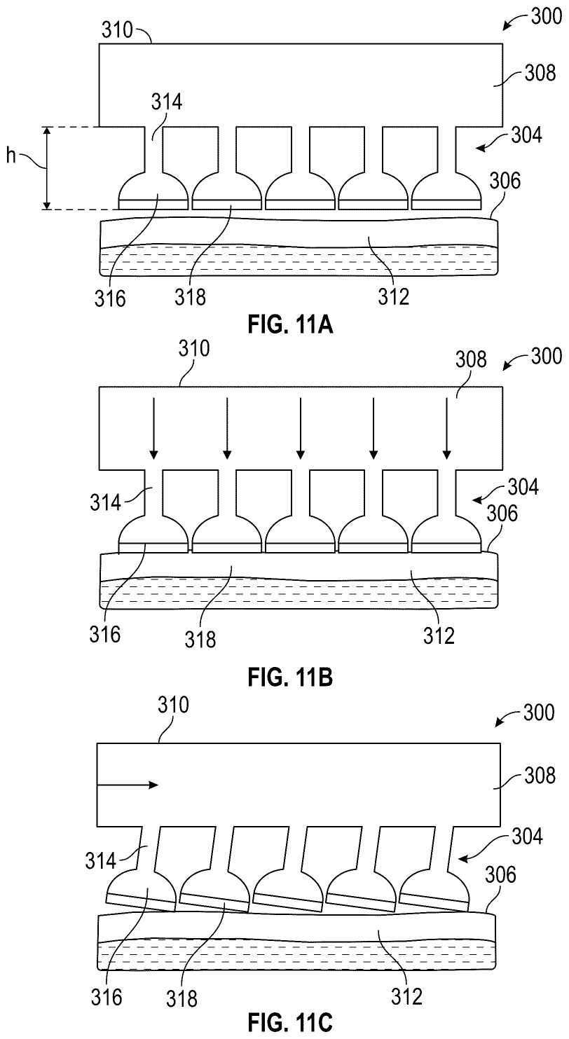

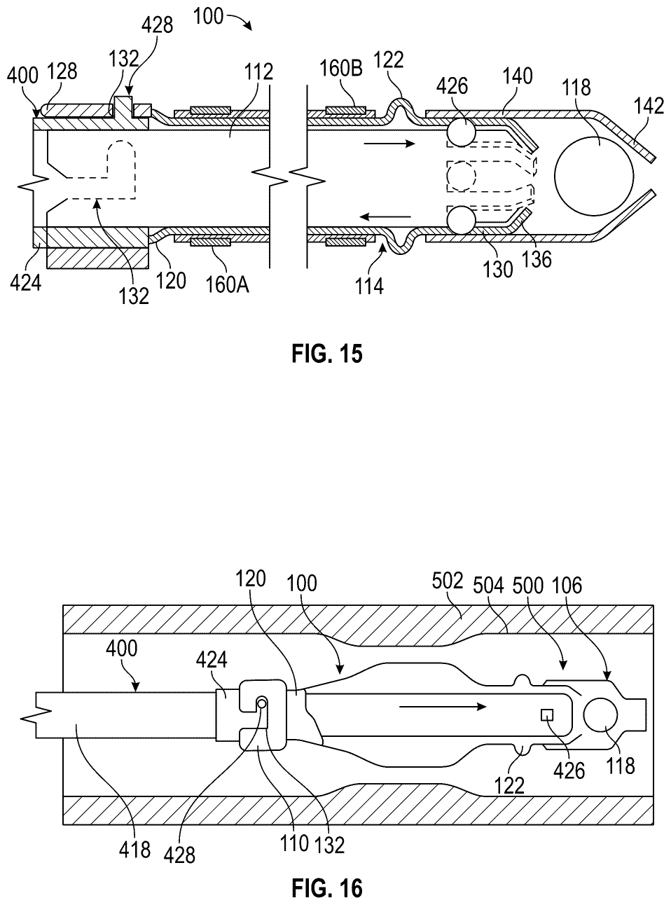

Membrane 108 can comprise a tubular body fitted around valve frame 114. In examples, membrane 108 can comprise a tubular body or sleeve that encircles valve frame 114. Membrane 108 can comprise a material that stretches. Thus, membrane 108 can be securely fitted around valve frame 114 and held in place by compressive forces when valve frame 114 is in an undeployed or collapsed stated. Membrane 108 can stretch further when valve frame 114 is radially expanded to a deployed state as described herein. Further description of the properties of membrane 108 are discussed with reference to FIG. 11A through FIG. 11C. Outer ring 126 and distal end ring 122 can prevent axial displacement of membrane 108 along valve frame 114. Thus, membrane 108 can be held in place without the use of any adhesives or glue or another positive attachment means, such as fasteners, hooks or the like. However, in various examples, positive attachment means, such as fasteners or adhesive can be used. For example, as shown in FIG. 9A and FIG. 15, band 160A and band 160B can be placed around membrane 108 to facilitate attachment of membrane 108 to valve frame 114. In examples, band 160A can be positioned proximate to proximal end ring 120 and band 160B can be positioned proximate to distal end ring 122. However, band 160A and band 160B can be located in other positions. In examples, only a single band can be used, or more than two bands can be used. In various configurations, membrane 108 can thus be allowed to slide against struts 124 between band 160A and band 160B as struts 124 are radially expanded and membrane 108 stretches.

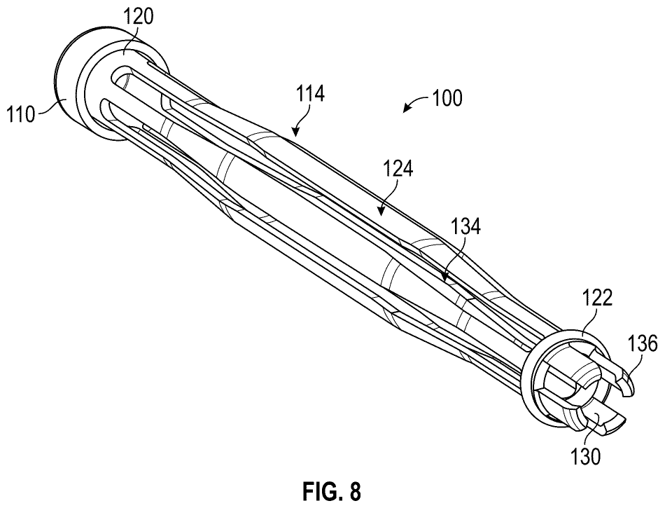

Struts 124 can be separated by slots 134. Struts 124 can form a portion of lumen 112 extending through valve frame 114. Struts 124 and slots 134 therebetween can be shaped to facilitate controlled deformation of valve frame 114 when subject to forces from delivery system 400 (FIG. 12A), as discussed with reference to FIG. 7 and FIG. 8, to transform valve from a collapsed or non-deployed state to an expanded or deployed state. The illustrated example of valve frame 114 comprises elongate struts having rectilinear cross-sectional shapes but, in other examples, other shaped struts, can be used, such as round, oval, hexagonal and octagonal.

Fingers 130 can extend distally from distal end ring 122. Fingers 130 can comprise a portion of the material of valve frame 114. Fingers 130 can be cantilevered from distal end ring 122 such that the proximal ends are supported by distal end ring 122 and the distal ends are deflectable in the radial direction. Further, the distal ends of fingers 130 can be bent or curved radially inward to form flanges 136. Flanges 136 can form a constriction in lumen 112 that prevents ball 118 from moving proximally further into valve frame 114. However, spacing between fingers 130 can allow fluid flow through valve frame 114 when ball 118 engages flanges 136.

During assembly, ball 118 can be positioned against flanges 136 and valve housing 116 can be positioned over fingers 130 to trap ball 118 therebetween. Valve housing 116 can comprise cylindrical body 140, neck 142 and inlet 144. Cylindrical body 140 can have an inner diameter sized to fit over fingers 130. Cylindrical body 140 and fingers 130 can be configured to form an interference fit to prevent or inhibit valve housing 116 from separating from valve frame 114 once assembled. However, valve housing 116 can be secured in place using mechanical fasteners, such as screws, or via metallurgical means, such as welding including laser welding. In examples, valve housing 116 can be attached using laser welding around the entirety of the circumference of valve housing 116.

Cylindrical body 140 can be pushed along fingers 130 to engage distal end ring 122 and can be welded in place, such as by the use of laser welding. Cylindrical body 140 can extend distally beyond fingers 130 to provide space for ball 118. Neck 142 can extend radially inward from cylindrical body 140 to provide a reduced diameter portion relative to lumen 112. Neck 142 can form a pocket to partially receive ball 118 and prevent ball 118 from passing distally through valve housing 116. Cylindrical body 140 and neck 142 can be sized to allow ball 118 to move axially between neck 142 and flanges 136. Thus, the distance between flanges 136 and inlet 144 can be greater than the diameter of ball 118. Inlet 144 can comprise an annular body having a reduced diameter that can allow fluids to enter or leave valve frame 114. Ball 118 can engage flush with neck 142 to prevent or inhibit flow of fluids through inlet 144.

Ball 118 can comprise a spherical body configured to fit within cylindrical body 140. In examples, ball 118 can be hard, such as by being fabricated from metal material such as stainless steel. In examples, ball 118 can be fabricated from plastic materials. In examples, ball 118 can be deformable. In examples, ball 118 can be solid or hollow.

With ball 118 trapped between inlet 144 and flanges 136, flow of fluid against ball 118 can operate valve assembly 106. Fluid can flow proximally (from right to left in FIG. 5) by displacement of ball 118 proximally to engage flanges 136 and thereby opening inlet 144. However, fluid is prevented from flowing distally (from left to right in FIG. 5) by displacement of ball 118 distally to seal against neck 142.

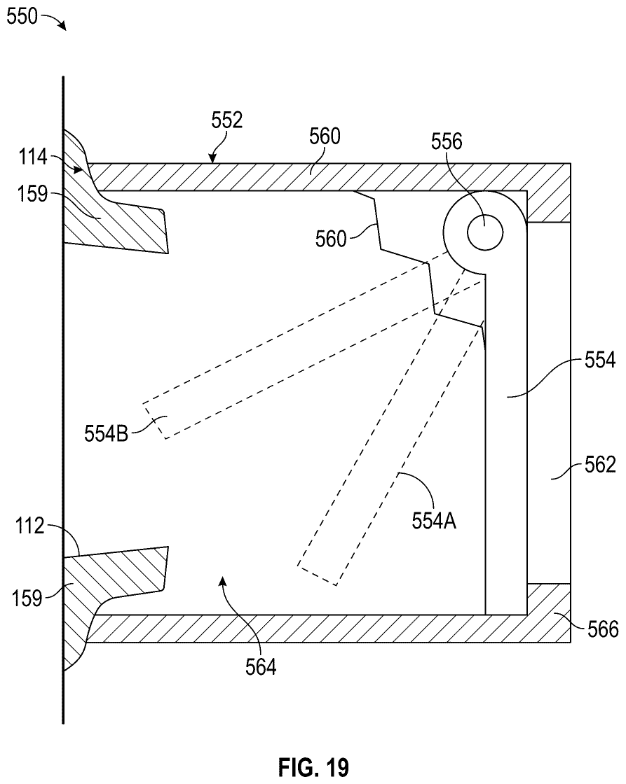

In the example of FIG. 5, valve assembly 106 comprises a ball valve. However, other types of valves can be used, particularly those that allow one-way flow and block flow in the opposite direction. For example, FIG. 19 describes a flapper type valve that can be used with the bronchial valve devices of the present disclosure. Other types of valves that can be used with the devices described herein comprise duckbill valves, butterfly valves, check valves, diaphragm valves, reed valves and the like.

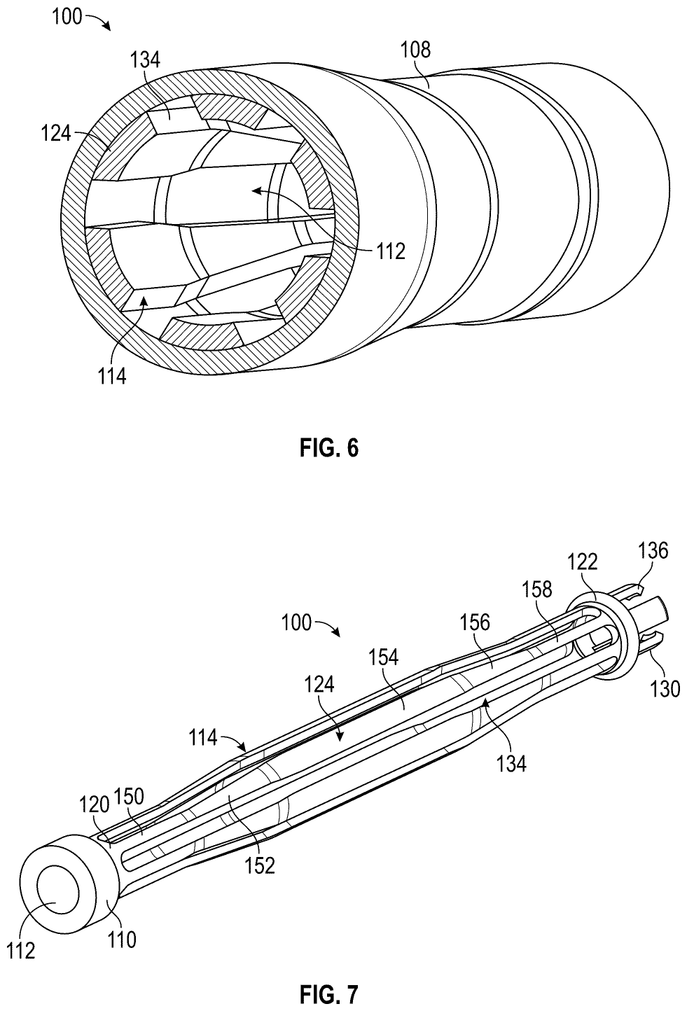

FIG. 6 is a perspective cross-sectional view of bronchial valve 100 of FIG. 4 taken along section 6-6 showing struts 124 and slots 134 of valve frame 114. FIG. 7 is a perspective view of proximal end 102 of valve frame 114 of bronchial valve 100 of FIG. 3 and FIG. 5. FIG. 8 is a perspective view of distal end 104 of valve frame 114 of bronchial valve 100 of FIG. 3 and FIG. 5. FIG. 6 through FIG. 8 are discussed concurrently.

In the illustrated example, valve frame 114 can comprise six struts 124 and six slots 134. However, fewer struts or a greater number of struts can be included.

Struts 124 can extend between proximal end ring 120 and distal end ring 122. Struts 124 can be shaped to induce radial outward expansion of valve frame 114 in a controlled manner. Each of struts 124 can comprise proximal portion 150, proximal tapered portion 152, middle portion 154, distal tapered portion 156 and distal portion 158.

Proximal portion 150 and distal portion 158 can be narrower in width than middle portion 154. Accordingly, proximal portion 150 and distal portion 158 can have smaller cross-sectional areas that middle portion 154. As such, proximal portion 150 and distal portion 158 can be more readily deformed than middle portion 154, and middle portion 154 can be straighter and stiffer to provide occlusion.

Proximal portion 150 and distal portion 158 can have diameters that are smaller than middle portion 154. Accordingly, proximal portion 150 and distal portion 158 can be positioned closer to central axis AA. As such, middle portion 154 can be displaced further radially outward than proximal portion 150 and distal portion 158 in order to help induce outward radial expansion.

Proximal tapered portion 152 and distal tapered portion 156 can be tapered to connect proximal portion 150 and distal portion 158 with middle portion 154. That is, the thickness of proximal tapered portion 152 and distal tapered portion 156 in the circumferential direction can diminish or grow as each portion extends axially. Specifically, the circumferential thickness of proximal tapered portion 152 can grow in the distal direction and the circumferential thickness of distal tapered portion 156 can grow in the proximal direction.

Proximal tapered portion 152 and distal tapered portion 156 can be angled, bowed or sloped to connect proximal portion 150 and distal portion 158 with middle portion 154. That is, the distance from central axis AA of proximal tapered portion 152 and distal tapered portion 156 in the radial direction can diminish or grow as each portion extends axially. Specifically, the radial height of proximal tapered portion 152 can grow in the distal direction and the radial height of distal tapered portion 156 can grow in the proximal direction.

In examples, each strut 124 can comprise a narrow and straight proximal portion, a tapered and included proximal portion, a wide and straight central portion, a tapered and declined distal portion and a narrow and straight distal portion.

In examples, proximal portion 150 and distal portion 158 can each comprise approximately fifteen percent of the length of valve frame 114, proximal tapered portion 152 and distal tapered portion 156 can each comprise approximately fifteen percent of the length of valve frame 114, and middle portion 154 can comprise approximately forty percent of the length of valve frame 114.

In examples, proximal portion 150 and proximal tapered portion 152 can together comprise approximately twenty-five percent of the length of valve frame 114, distal portion 158 and distal tapered portion 156 can together comprise approximately twenty-five percent of the length of valve frame 114, and middle portion 154 can comprise approximately fifty percent of the length of struts 124.

Middle portion 154 can be long relative to proximal portion 150, proximal tapered portion 152, distal portion 158 and distal tapered portion 156 in order to provide a large surface area for contacting tissue when valve frame 114 is expanded. Thus, as opposed to other valves in the art that have curved surfaces that that engage tissue only along a narrow strip at their largest diameter rim, valve frame 114 can provide larger engagement area with tissue to help prevent migration or movement of bronchial valve 100 when implanted.

As discussed herein, proximal end ring 120 and distal end ring 122 can be brought closer to each other by mechanical application of force to cause struts 124 to flex or bend outwardly. In particular, middle portion 154 can be displaced radially outwardly further away from central axis AA than the position of FIG. 5. Due at least in part to the shaping of struts 124, middle portion 154 can be radially extended with minimal induced curvature therein. In particular, valve frame 114 can be bent between proximal portion 150 and proximal tapered portion 152, between proximal tapered portion 152 and middle portion 154, between middle portion 154 and distal tapered portion 156 and between distal tapered portion 156 and distal portion 158. Struts 124 can be equidistant from axis central axis AA so that uniform expansion occurs, e.g., valve frame 114 can maintain a generally circular cross-sectional profile in the radially collapsed state and in any radially expanded state.

FIG. 9A is a cross-sectional view of bronchial valve 100 of the present disclosure including struts 124 in an axial configuration and in an elongate, undeployed state. Bronchial valve 100 can extend from coupler 110 to valve assembly 106. Struts 124 and slots 134 can extend axially from proximal end ring 120 at coupler 110 to distal end ring 122 at valve assembly 106 parallel to central axis AA. Slots 134 can extend from proximal portion 150 to distal portion 158. However, slots 134 can be axially shorter, such as by extending from proximal tapered portion 152 to distal tapered portion 156 or being located within middle portion 154. Additionally, in examples, slots 134 can be axially positioned asymmetrically anywhere on or between proximal portion 150 and distal portion 158. Slots 134 can have the inverse shape of struts 124. In examples, slots 134 can comprise simple straight slots with rounded ends.

Side portions of struts 124 can be flat and can extend generally perpendicular to central axis AA. In particular, proximal portion 150, middle portion 154 and distal portion 158 can have sidewalls connecting the inner diameter surface and the outer diameter surface that are parallel to central axis AA. In other words, slots 134 can extend straight into valve frame 114 so that the cross-sectional area of slots 134 is the same at the inner diameter surface and the outer diameter surface of valve frame 114.

In the undeployed state of FIG. 9A, proximal portion 150 and distal portion 158 can be located distance D1 away from central axis AA, and middle portion 154 can be located distance D2 away from central axis AA, with distance D2 being greater than distance D1. Proximal tapered portion 152 and distal tapered portion 156 can be inclined and declined (relative to a proximal-to-distal direction) to connect middle portion 154 to proximal portion 150 and distal portion 158. Furthermore, in the undeployed state, the angles between proximal portion 150 and proximal tapered portion 152 and distal portion 158 and distal tapered portion 156 on the exterior of valve frame 114 can comprise and obtuse angles in the range of approximately one-hundred-seventy-five degrees to approximately one-hundred-sixty degrees.

FIG. 9B is a cross sectional view of bronchial valve 100 of FIG. 9A in a contracted, deployed state. As discussed below, an axial mechanical force can be applied to distal end ring 122 parallel to central axis AA to cause distal end ring 122 to be moved closer to proximal end ring 120. This force can cause struts 124 to undergo deformation. Specifically, struts 124 can undergo controlled deformations where further bending occurs between portions of struts that are already bent or angled relative to each other. In particular, valve frame 114 can be bent between proximal portion 150 and proximal tapered portion 152, between proximal tapered portion 152 and middle portion 154, between middle portion 154 and distal tapered portion 156 and between distal tapered portion 156 and distal portion 158.

In the deployed state of FIG. 9B, proximal portion 150 and distal portion 158 can remain at distance D1 away from central axis AA, and middle portion 154 can be moved to distance D3 away from central axis AA, wherein distance D3 is greater than distance D2 of FIG. 9A. As mentioned, middle portion 154 can be uniformly expanded to distance D3 across its length. Proximal tapered portion 152 and distal tapered portion 156 can be inclined and declined (relative to a proximal-to-distal direction) to connect middle portion 154 to proximal portion 150 and distal portion 158. Furthermore, in the undeployed state, the angles between proximal portion 150 and proximal tapered portion 152 and distal portion 158 and distal tapered portion 156 on the exterior of valve frame 114 can comprise and obtuse angles in the range of approximately ninety degrees to approximately one-hundred-twenty degrees.

Axial contraction of valve frame 114 and the resultant expansion of struts 124 to an enlarged outer profile can allow for bronchial valve to be adjusted to fit within different sized anatomic ducts. Valve frame 114 can be expanded to an infinitesimal number of expanded states. Specifically, a surgeon or operator of delivery system 400 (FIG. 12A and FIG. 13) to control the amount of axial contraction and radial expansion. Thus, the bronchial valve 100 can be expanded more or less to fit into larger or smaller anatomic ducts. Furthermore, as discussed herein, the use of microfibers 304 can facilitate use with anatomic ducts of different cross-sectional size and shape.

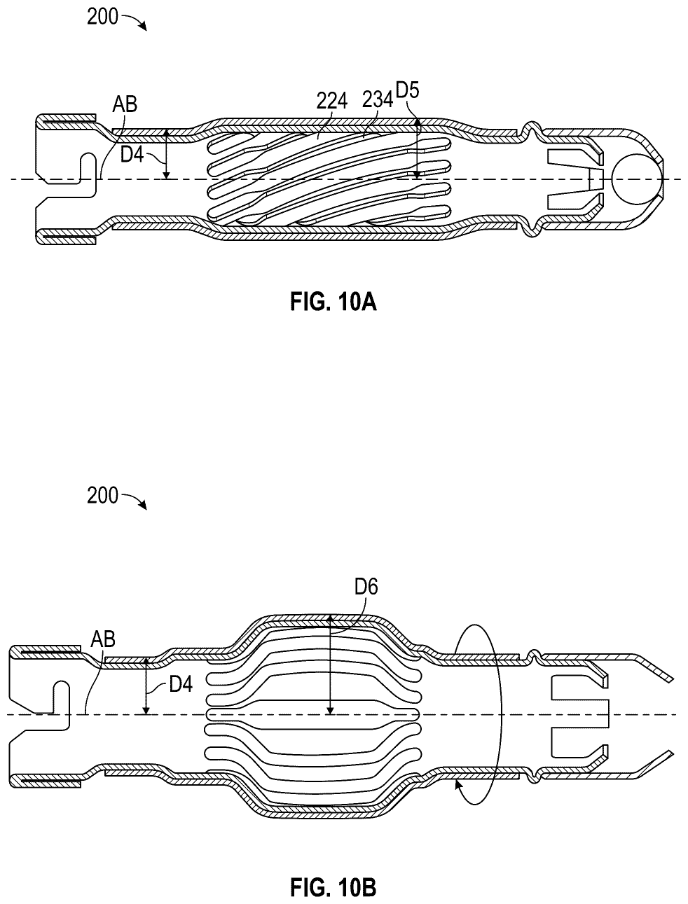

FIG. 10A is a cross-sectional view of bronchial valve 200 of the present disclosure including struts 224 in a spiral configuration in an elongate, undeployed state. Bronchial valve 200 can be configured similarly as bronchial valve 100 except that struts 224 can extend relative to central axis AB in a spiral manner rather than a linear manner like struts 124. Bronchial valve 200 can have similar components as bronchial valve 100 that are designated with two-hundred series reference numbers instead of one-hundred series reference numbers. Bronchial valve 200 can comprise proximal end 202, distal end 204, membrane 208, internal lumen 212, ball 218, fingers 230, keyway 232 and flanges 236.

Bronchial valve 200 can extend from coupler 210 to valve assembly 206. Struts 224 and slots 234 can extend from proximal end ring 220 at coupler 210 to distal end ring 222 at valve assembly 206 at angles to central axis AB. Slots 234 can be located within middle portion 254. However, slots 234 can be axially longer to extend from proximal portion 250 to distal portion 258 or extend from proximal tapered portion 252 to distal tapered portion 256. Additionally, in examples, slots 234 can be axially positioned asymmetrically anywhere on or between proximal portion 250 and distal portion 258. Slots 234 can have the inverse shape of struts 224. In examples, slots 234 can comprise simple straight slots with rounded ends.

Side portions of struts 224 can be flat and can extend at angles to central axis AB. In particular, proximal portion 250, middle portion 254 and distal portion 258 can have sidewalls connecting the inner diameter surface and the outer diameter surface that are angled relative to central axis AB. In examples, the sidewalls of proximal portion 250, middle portion 254 and distal portion 258 can be angled in the range of approximately twenty-degrees to approximately forty degrees relative to axis AB. However, the sidewall of struts 224 can extend straight into valve frame 214 so that the cross-sectional area of slots 234 is the same at the inner diameter surface and the outer diameter surface of valve frame 214.

In the undeployed state of FIG. 10A, proximal portion 250 and distal portion 258 can be located distance D4 away from central axis AB and middle portion 254 can be located distance D5 away from central axis AB, with distance D5 being greater than distance D4. Proximal tapered portion 252 and distal tapered portion 256 can be inclined and declined (relative to a proximal-to-distal direction) to connect middle portion 254 to proximal portion 250 and distal portion 258. Furthermore, in the undeployed state, the angles between proximal portion 250 and proximal tapered portion 252 and distal portion 258 and distal tapered portion 256 on the exterior of valve frame 214 can comprise and obtuse angles in the range of approximately one-hundred-seventy-five degrees to approximately one-hundred-sixty degrees.

FIG. 10B is a cross sectional view of bronchial valve 200 of FIG. 10A in a contracted, deployed state. As discussed below, an axial mechanical force can be applied to distal end ring 222 parallel to central axis ABA to cause distal end ring 222 to be moved closer to proximal end ring 220. This force can cause struts 224 to undergo deformation. Specifically, struts 224 can undergo controlled deformations where further bending occurs between portions of struts that are already bent or angled relative to each other. In particular, valve frame 214 can be bent between proximal portion 250 and proximal tapered portion 252, between proximal tapered portion 252 and middle portion 254, between middle portion 254 and distal tapered portion 156 and between distal tapered portion 256 and distal portion 258. Deployment of valve frame 214 can be performed using a rotary action. For example, lock ring 424 (FIG. 13) can be rotated by attachment knob 410 (FIG. 13) to cause unwinding of struts 224. In such configurations, handle 402 can be adapted to allow for resulting relative axial movement between ends of valve frame 214, such as by allowing locking knob 406 to translate axially.

In the deployed state of FIG. 10B, proximal portion 250 and distal portion 258 can remain at distance D4 away from central axis AB and middle portion 254 can be moved to distance D6 away from central axis AB, with distance D6 being greater than distance D5 of FIG. 10A. Middle portion 254 can be uniformly expanded to distance D6 across its length. Proximal tapered portion 252 and distal tapered portion 256 can be inclined and declined (relative to a proximal-to-distal direction) to connect middle portion 254 to proximal portion 250 and distal portion 158. Furthermore, in the undeployed state, the angles between proximal portion 250 and proximal tapered portion 252 and distal portion 158 and distal tapered portion 256 on the exterior of valve frame 214 can comprise and obtuse angles in the range of approximately ninety degrees to approximately one-hundred-twenty degrees.

As can be seen in FIG. 10B, struts 224 can unwind or straighten when moving from the undeployed state to the deployed state. Thus, in the deployed state of FIG. 10B, struts 224 and slots 234 can be parallel to central axis AB. For the illustrated spiral shape of struts 224, distal end 204 can rotate clockwise relative to proximal end 202 when looking along valve frame 214 in the proximal-to-distal direction. The spiral shape of struts 224 can facilitate a controlled expansion of valve frame 214.

FIG. 11A is a schematic close-up view of membrane 300 of the present disclosure having outer surface 302 with microfibers 304 positioned proximate to surface 306 to which microfibers 304 are to be adhered. Membrane 300 can comprise base layer 308 having outer surface 302 and inner surface 310. Inner surface 310 can be positioned adjacent struts 124 of valve frame 114. Outer surface 302 can be positioned radially outwardly to face toward surface 306. In examples, surface 306 can comprise a surface of tissue 312 of an anatomic passage in which bronchial valve 100 is to be deployed.

Base layer 308 can form a layer or sleeve surrounding at least some of the axial length of valve frame 114. Base layer 308 can form a barrier that prevents fluid, e.g., breathed air, blood, mucus, etc., from flowing around bronchial valve 100, e.g., out of lumen 112 (FIG. 1). Furthermore, base layer 308 can prevent the flow of fluid between the outer surface of valve frame 114 (FIG. 3) and surface 306.

Microfibers 304 can comprise stem 314, head 316 and face 318. Stem 314 can comprise an elongate body extending from outer surface 302. In examples, stem 314 can be integral or monolithic with base layer 308. Stem 314 can comprise a cylindrical body having a round cross-sectional profile, but can have other cross-sectional profiles, such as square, hexagonal, octagonal, oval or elliptical. Stem 314 can be configured to be flexible and stretchable. Microfibers 304 can behave axial symmetry about the longitudinal axes of stems 314.

Head 316 can comprise a body having a larger cross-sectional profile than stem 314. In examples, head 316 can be integral or monolithic with stem 314. Head 316 can comprise a cup-shaped body having a round cross-sectional profile, but can have other cross-sectional profiles, such as square, hexagonal, octagonal, oval or elliptical. Head 316 can provide a surface area to support face 318. The sides of head 316 can have different shapes. In the illustrated example, the sides of head 316 are linear. In other examples, the sides of head 316 can be concave or convex with respect to the stem axial direction and face 318.

Face 318 can comprise the outward-facing portion of head 316. In examples, face 318 can be integral or monolithic with head 316. Face 318 can comprise a flat surface or a concave surface having a round cross-sectional profile, but can have other cross-sectional profiles, such as square, hexagonal, octagonal, oval or elliptical. Face 318 can be configured to adhere to another surface. Specifically, face 318 can include adherence features that allow head 316 to adhere to a surface, whether wet or dry. The edge of face 318 can be pointed, rounded or flattened. Face 318 will typically not adhere to a surface unless first pressed into such surface with some amount of force, thus allowing face 318 to freely slide against surfaces when not in use.

Microfibers 304 can increase the gripping, frictional and adhesive performance of bronchial valve 100. In particular, the enlarged cross-sectional area of head 316 relative to stem 314 can increase contact area and increase the pull-off capability of microfibers 304.

In examples, microfibers 304 can comprise biostable materials such as carbon, silica, polyester, nylon, polyamides, polyolefins, polyurethanes and combinations of these and other materials. In examples, base layer 308 can comprise nylon, polyamide, polyurethane, polyimide, polyethylene terephthalate (PET), polytetrafluroethylene (PTFE), fluorinated ethylene propylene, a fabric of woven polyester fibers such as Dacron® fibers (E.I. Du Pont De Nemours & Co., Inc.), or other medically acceptable materials. In examples, microfibers 304 and base layer 308 can comprise the same material or different materials. Stems 314 can be attached to base layer 308 by bonding or with an adhesive or can be formed integrally, such as by casting, with base layer 308.

In additional examples, microfibers 304 and base layer 308 can be produced from any moldable plastic, including thermosets, formaldehyde resins, polyurethanes (PU), unsaturated polyester resins (UP), vinylester resins (VE), phenacrylate resins, vinylester urethanes (VU), epoxy resins (EP), diallyl phthalate resins, allyl esters (PDAP), silicone resins (Si), rubbers, thermoplastics, polyolefins (PO), polypropylene copoplymers, polybutene (PB, PIB), higher poly-a-olefins (PMP, PDCPD), styrene polymers, vinyl polymers, fluoropolymers, polyacryl- and methacryl copolymers, polyamides (PA), liquid crystalline polymers (LCP), biopolymers, naturally occurring polymers and derivates, polymer ceramics, polysilicooxoaluminate (PSIOA), thermoplastic elastomers and others.

In examples, microfibers 304 can be fabricated form polyurethane having a 60 Shore A hardness. In other examples, microfibers 304 can have a hardness ranging from 10 Shore A to 100 Shore D.

Microfibers 304 and base layer 308 can be fabricated using injection molding, compression molding, thermoforming, casting, coating processes, blow molding, vinyl dispersions such as dip molding, and composite manufacturing techniques involving molds, such as autoclave processing, bag molding and the like.

In examples, microfibers 304 can be configured according to the structures described in Pat. No. 8,720,047 to Hulseman et al., titled “Method for making Microstructured Objects,” the contents of which are incorporated herein by this reference.

In examples, microfibers 304 can be configured according to the structures described in Pat. No. 11,613,674 to Sitti et al., titled “Microfibers with Mushroom-Shaped Tips for Optimal Adhesion,” the contents of which are incorporated herein by this reference. As described therein, microfibers 304 can comprise microfibers or nanofibers, including those with characteristic geometries ranging from 0.2-500 nm and 0.2-500 μm in height h. Each of microfibers 304 can be, in examples, part of a microfiber array of synthetic polyurethane mushroom-shaped fibers, also known as “gecko feet” fibers. Each of microfibers 304 can have a 4 μm stalk radius (e.g., the radius of stem 314), an 8 μm tip radius (e.g., the radius of head 316), and a 20 μm length or height h (Scale bar: 100 μm). In examples, microfibers 304 can comprise microfibers commercially available from Setex Technologies, Inc. of Pittsburgh, Pennsylvania.

As shown in FIG. 11A, heads 316 of microfibers 304 can be positioned adjacent to surface 306 by positioning bronchial valve 100 adjacent to surface 306.

FIG. 11B is a schematic close-up view of membrane 300 of FIG. 11A being brought into engagement with surface 306. Specifically, faces 318 of heads can be brought into contact with surface 306. Thus, the adherence features of faces 318 can grab ahold of surface 306. As mentioned, activation of the adherence features of faces 318 can be done by pressing faces 318 into a surface perpendicularly to faces 318, e.g., along the axes of stems 314.

FIG. 11C is a schematic close-up view of membrane 300 of FIG. 11B showing microfibers 304 stretching as surface 306 is displaced relative to base layer 308. For example, a side-to-side or shear force can be applied to base layer 308 causing lateral movement of bronchial valve 100 relative to tissue 312. Stems 314 can stretch to allow relative displacement between tissue 312 and base layer 308. However, heads 316 can remain attached to surface 306 of tissue 312 via operation of the adherence features of faces 318. Thus, microfibers 304 can prevent axial migration of base layer 308, and valve frame 114 therein, along surface 306. However, if sufficient amount of force is applied to faces 318, particular perpendicular thereto, heads 315 can detach from surface 306.

Microfibers 304 can be used to accommodate differences in the expanded size of valve frame 114 and the anatomic duct in which it is deployed. For example, microfibers 304 can stretch to accommodate differences in the expanded size of valve frame 114 and the anatomic duct. Thus, if valve frame 114 slightly contracts, e.g., due to relaxation, microfibers 304 can stretch to maintain engagement between bronchial valve 100 and the anatomic duct. Additionally, if the anatomic duct were to stretch for some reason, microfibers 304 can stretch to maintain engagement between bronchial valve 100 and the anatomic duct.

FIG. 12A is a cross-sectional view of delivery system 400 comprising handle 402 and shaft 404. Handle 402 can comprise locking knob 406, deployment knob 408 and attachment knob 410. FIG. 12B is cross-sectional view of handle 402 of FIG. 12A with locking knob 406 in a retracted state to pull cable 412. FIG. 12C is a cross-sectional view of handle 402 of FIG. 12B with deployment knob 408 actuated to retract locking knob 406 and cable 412. FIG. 13 is a partial cross-sectional view of coupling mechanism 405 of delivery system 400 of FIG. 12A through FIG. 12C.

Handle 402 can be connected to locking knob 406, deployment knob 408 and attachment knob 410. Locking knob 406 can be connected to cable 412 and screw 114. Deployment knob 408 can be engaged with screw 414, which can be connected to inner tube 416, which can comprise a flexible stainless steel braided tube. Attachment knob 410 can be connected to outer tube 418, which can comprise a catheter tube. Cable 412 can be connected to locking mechanism 420 of FIG. 14 within distal tip 422. Inner tube 416 can be connected to distal tip 422. In examples, inner tube 416 can be arranged in section joined by coupler 429 (FIG. 13). Outer tube 418 can be connected to lock ring 424 disposed about distal tip 422. Distal tip 422 can include locking balls 426 that engage with locking mechanism 420 of FIG. 14. Lock ring 424 can include locking pegs 428. In example, four locking pegs 428 can be provided spaced approximately ninety-degrees apart. In example, four locking balls 426 can be provided approximately ninety-degrees apart.

As discussed herein, locking knob 406 can be used to control the radial position of locking balls 426 to facilitate attachment to valve frame 114 in between fingers 130, sch as by pulling on cable 412, deployment knob 408 can be used to axially contract or axially extend valve frame 114 (FIG. 3) between proximal end ring 120 and distal end ring 122 by axially contracting and extending the position of locking balls 426 relative to locking pegs 428, such as by pulling on inner tube 416, and attachment knob 410 can be used to control the rotational position of lock ring 424 for attaching locking pegs 428 to valve frame 114 at keyway 132, such as by rotating outer tube 418, as explained in greater detail below.

FIG. 14 is a schematic cross-sectional view of locking mechanism 420 within distal tip 422. Locking mechanism 420 can comprise sliding block 430, cap 432, biasing block 434 and spring 436. Sliding block 430 can comprise a cylindrical body having a plurality of sockets 440 to receive locking balls 426. Sockets 440 can comprise elongate slots having widths approximately the diameter of locking balls 426 and lengths longer than the diameter of locking balls 426. Cap 432 can be attached to sliding block 430 and can define distal ends of sockets 440. As such, locking balls 426 can be positioned within sockets 440 and can axially roll therewithin. Biasing block 434 can be positioned in sockets 440 to bias locking balls 426 toward sliding block 430, away from cap 432. The distal ends of sockets 440 can be curved or sloped. Similarly, the proximal ends of biasing block 434 can be curved or sloped oppositely as the distal ends of sockets 440. Thus, the distal ends of sockets 440 and the proximal ends of biasing block 434 can form a cup, depression or concavity to receive locking balls 426. One of more of spring 436 can bias or push biasing block 434 in the proximal direction so that locking balls 426 are pushed radially outward. Locking balls 426 can remain in a radially outward position, as shown, except when sliding engagement with a valve device causes temporary radially inward movement of locking balls 426 and compression of spring 436. Sliding block 430 can be positioned distally with locking knob 406 in its distal-most position to unlock locking balls 426, thereby allowing locking balls 426 to move into a radially retracted position to allow for a valve device to slide over locking balls 426. For example, when fingers 130 slide over locking balls 426, locking balls 426 can be pushed radially inward and cause biasing block 434 to move distally and compress spring 436 until locking balls 426 find the slots between fingers 130. To lock locking balls 426 into the radially outward position between fingers 130, sliding block 430 can be retracted proximally using locking knob 406 to pull cap 432 closer to biasing block 434, compressing spring 436, and prevent biasing block 434 from moving distally, thereby immobilizing locking balls 426 into a radially outward position to maintain coupling to the valve device. Proximal movement of cap 432 can push spring 436 proximally, which can cause biasing block 434 to move proximally to push locking balls 426 against ports 480 (FIG. 13). Thus, operation of locking knob 406 can control the distance between cap 432 and ports 480, thereby controlling the amount of runway that biasing block 434 has to move. Likewise, operation of locking knob 406 can cause relative movement between cable 412 and inner tube 416.

Returning to FIG. 12A and FIG. 13, locking knob 406 can comprise knob 450, neck 452, threaded stem 454 and socket 456. Handle 402 can include keyway 457 to receive screw 414 to prevent screw 414 from rotating with deployment knob 408. Screw 414 can include cup 458 having internally threaded bore 459 and radially outer keys for mating with keyway 457. Threaded stem 454 of locking knob 406 can be threadedly engaged with threaded bore 459. Cable 412 can be inserted into socket 456 within threaded stem 454. The proximal end of cable 412 can include head 462 that can be located in enlarged portion 464 of socket 456. Cable 412 can extend from socket 456. Screw 414 can be located within channel 470 of handle 402. Screw 414 can include internal passage 472 through which cable 412 can extend. Deployment knob 408 can be located in socket 474 within handle 402. Deployment knob 408 can include radially inner threading that can engage with radially outer threading on screw 414. Cable 412 can extend from screw 414, through attachment knob 410 and into outer tube 418. Inner tube 416 can extend from screw 414, through attachment knob 410 and into outer tube 418.

Distal tip 422 can comprise a cylindrical body having internal space 476 in which locking mechanism 420 (FIG. 14) can be located. Cable 412 can extend into distal tip 422 to connect with locking mechanism 420. Specifically, the distal end of cable 412 can connect to sliding block 430. The distal end of inner tube 416 can connect to distal tip 422. Lock ring 424 can comprise an annular body having passage 478 through which distal tip 422 can extend. Distal tip 422 can include ports 480 for locking balls 426.

FIG. 15 is a side cross-sectional view of bronchial valve 100 of the present disclosure schematically showing locking balls 426 of distal tip 422 and locking mechanism 420 of FIG. 14 inserted therein. Description of the operation of delivery catheter is described with reference to FIG. 12A through FIG. 12C, FIG. 13, FIG. 14 and FIG. 15 concurrently.

With reference to FIG. 15, in order to deploy bronchial valve 100, distal tip 422 can be inserted into lumen 112 at coupler 110. Specifically, distal tip 422 can be inserted into bronchial valve 100 so that locking balls 426 align with fingers 130 and locking pegs 428 align with keyway 132. With reference to FIG. 12A, locking knob 406 can be rotated to allow for slack within cable 412 by moving sliding block 430 distally (to the right in FIG. 12A) to move sliding block 430 into a distal position and allowing biasing block 434 to have distal runway within sockets 440 to move distally when a radially inward force is applied to locking balls 426, thereby allowing locking balls 426 to drop out of ports 480. As shown in FIG. 12A, cup 458 of screw 414 can be engaged with neck 468 on handle 402 to move sliding block 430 in the maximal distal position to remove tension in cable 412. With reference to FIG. 14, once locking balls 426 are located between fingers 130, spring 436 can push biasing block 434 proximally to maintain locking balls in a biased, but not locked, radially outward state. With reference to FIG. 15, distal tip 422 can be inserted so that locking pegs 428 of lock ring 424 align with keyway 132. Locking pegs 428 can be inserted axially into proximal portions of keyway 132. Thereafter, attachment knob 410 (FIG. 12A) can be rotated to cause rotation of outer tube 418 and lock ring 424. Rotation of lock ring 424 can cause locking pegs 428 to move into radially extending distal portions of keyway 132. As such, valve frame 114 of bronchial valve 100 can be axially locked with delivery system 400 such that axial movement of handle 402 can additionally provide axial movement of bronchial valve 100.

With reference to FIG. 12B, with locking pegs 428 locked into place, locking mechanism 420 can be operated to lock the distal portion of bronchial valve 100 to delivery system 400 by locking the radial position of locking balls 426. Knob 450 of locking knob 406 can be rotated to move threaded stem 454 proximally (to the left in FIG. 12B) within threaded bore 459, thereby moving sliding block 430 proximally and away from biasing block 434. As shown in FIG. 12B, the distal end of threaded stem 454 can be retracted distance D1 from the base of cup 458. With reference to FIG. 13, proximal movement of sliding block 430 can cause a head of a fastener comprising cap 432 to move closer to biasing block 434, compressing spring 436, thereby preventing biasing block 434 from moving distally. Thus, locking balls 426 can be pushed radially outward into ports 480. Locking balls 426 can thus be wedged into engagement with ports 480 by a ramped surface of biasing block 434. Ports 480 can be slightly smaller than the diameter of locking balls 426 to allow locking balls 426 to partially extend from ports 480 without being able to to pass therethrough. Portions of locking balls 426 extending from ports 480 can be positioned between adjacent fingers 130. Additionally, locking balls 426 can be positioned axially downstream of material of valve frame 114 between fingers 130 proximate to distal end ring 122. Thus, locking balls 426 can be constrained from moving axially in the proximal direction by valve frame 114. Thus, the distal end of bronchial valve 100 can be attached to delivery system 400.

With both the proximal end of bronchial valve 100 attached to delivery system 400 at locking pegs 428 and the distal end of bronchial valve 100 attached to delivery system 400 at locking balls 426, delivery system 400 can be used to insert bronchial valve 100 into an anatomic passageway, such as a passageway in a lung. Once at a desired anatomical location, deployment knob 408 can be rotated to cause screw 414 and locking knob 406 to move proximally, further pulling on cable 412. As shown in FIG. 12C, the bottom of cup 458 can be moved distance D2 away from neck 468. This further proximal movement of can cause locking balls 426 to move closer to locking pegs 428 to radially expand bronchial valve 100, as explained with reference to FIG. 16. Movement of deployment knob 408 can cause cable 412 and inner tube 416 to move together. In other words, operation of deployment knob 408 can cause the entire locking mechanism 420, e.g., sliding block 430, locking balls 426, biasing block 434, spring 436, cable 412, and locking knob 406, to move proximally.

FIG. 16 is a cross-sectional view of bronchial valve 100 of the present disclosure loaded onto delivery system 400 and inserted into anatomic passage 500. Anatomic passage 500 can comprise duct wall 502 and duct surface 504. With bronchial valve 100 positioned in a desired location, deployment knob 408 can be actuated to radially expand valve frame 114 to immobilize bronchial valve 100 in the anatomic passageway. Deployment knob 408 can be rotated to induce rotation of screw 414. In particular, deployment knob 408 can be actuated to pull inner tube 416 in the distal direction. Movement of inner tube 416 can also move distal tip 422 proximally with locking balls 426. Proximal movement of locking balls 426 can cause locking balls 426 to move closer to locking pegs 428. Thus, as the distance between locking pegs 428 and locking balls 426 decreases, compressive force can be applied to valve frame 114. The application of compressive force to valve frame 114 can cause radial expansion of valve frame as described herein. Middle portion 154 can move radially outward to contact duct wall 502. An operator of delivery system 400 can control the amount of expansion of valve frame to a desired radial size in order to fit within a specific anatomic duct.

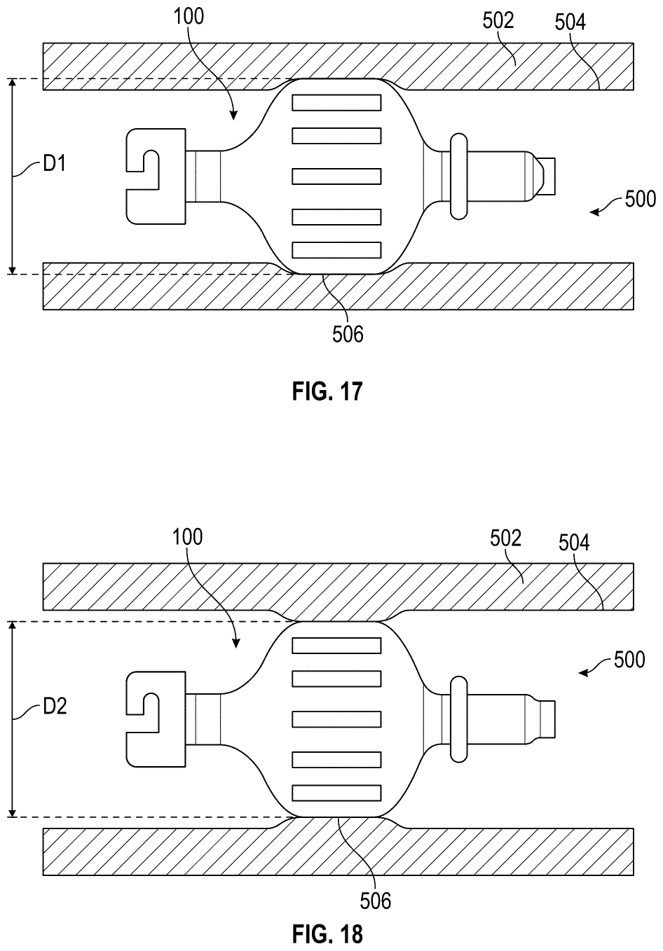

FIG. 17 is a schematic side view of bronchial valve 100 of the present disclosure deployed within anatomic passage 500 in an over-expanded state. Bronchial valve 100 can be expanded to diameter D1 which can be larger than the diameter of anatomic passage 500. Over expansion of bronchial valve 100 can cause undesirable effects in duct wall 502. For example, expansion of bronchial valve 100 can cause depression 506 to form within duct surface 504 of duct wall 502. Depression 506 can cause strain and stress within the tissue forming duct wall 502. With the valves of the present disclosure deployment of a bronchial valve that is too large for the anatomic duct in which it is deployed can be avoided because diameter D1 of bronchial valve 100 can be adjusted by a user to match the diameter of the anatomic duct it is being used with.

FIG. 18 is a schematic side view of bronchial valve 100 of the present disclosure deployed within anatomic passage 500 in an under-expanded state. Bronchial valve 100 can be expanded to diameter D2 (less than D1) which can be smaller than the diameter of anatomic passage 500. Under expansion of bronchial valve 100 can be undesirable with conventional bronchial valves because tissue barbs used therewith to hook such valves into tissue to prevent axial displacement can induce strain and stress within the tissue forming duct wall 502. For example, under-expansion of bronchial valve 100 can cause plateau 508 to form within duct surface 504 of duct wall 502. However, the present disclosure formation of localized stress and strain from barbs can be avoided by the use of microfibers 304. Specifically, microfibers 304 can minimize the formation of plateau 508 because stems 314 can stretch. Furthermore, with the valves of the present disclosure deployment of a bronchial valve that is too small for the anatomic duct in which it is deployed can be avoided because diameter D1 of bronchial valve 100 can be adjusted by a user to match the diameter of the anatomic duct it is being used with. As mentioned previously, microfibers 304 can additionally make-up for or offset effects of strain relaxation in bronchial valve 100 where expansion of valve frame 114 slightly un-expands so that D2 slightly shrinks. Thus, microfibers 304 of the present disclosure will help ensure adhesion or attachment of bronchial valve 100 even if the valve is not fully expanded to press into duct surface 504.

FIG. 19 is a cross-sectional view of flapper valve 550 suitable for use with the valves of the present disclosure. Flapper valve 550 can comprise housing 552, door 554, hinge 556 and spring 558. Housing 552 can be attached to valve frame 114. In particular, valve frame 114 can include flange 159 to which housing 552 can be joined. Housing 552 can comprise cylindrical body 560 having opening 562 and passage 564. Opening 562 can be surrounded by flange 566.

Flapper valve 550 can be used in place of ball 118 and valve housing 116 as valve assembly 106. As such, flange 159 of valve frame 114 can comprise fingers 130. However, in examples, fingers 130 can be replaced by a hoop or ring forming flange 159.

Housing 552 can be slipped over flange 159 and attached thereto using any suitable means, such as mechanical fasteners or metallurgical bonding. Thus, passage 564 can be placed in fluid communication with lumen 112 within valve frame 114. Door 554 can be attached to housing 552 via hinge 556. Hinge 556 can allow door 554 to rotate relative to housing 552. Door 554 can be configured to rotate approximately ninety degrees within housing 552. Spring 558 can be used to bias door 554 to a closed position. In the closed position, door 554 can engage with flange 566. A seal can be formed between door 554 and flange 566 to prevent or inhibit fluid flow therebetween. In examples, a seal, such as a rubber O-ring seal can be provided on flange 566 or door 554 to facilitate a sealed engagement.

In use, if fluid flows into passage 564 from lumen 112, the fluid can push against door 554, maintaining door 554 in the closed and sealed position. Flapper valve 550 can be positioned on valve frame 114 to prevent inhaled air flowing from lumen 112 from exiting flapper valve 550. However, if fluid flows into engagement with the exterior side of door 554 (the right side of door in FIG. 19), the fluid flow can overcome the force of spring 558 and push door 554 to an open position, as indicated by door 554A and 554B shown in dashed lines. Thus, flapper valve 550 can be positioned on valve frame 114 to allow exhaled air flowing from downstream of door 554 to pass through flapper valve 550 and exit from bronchial valve 100.

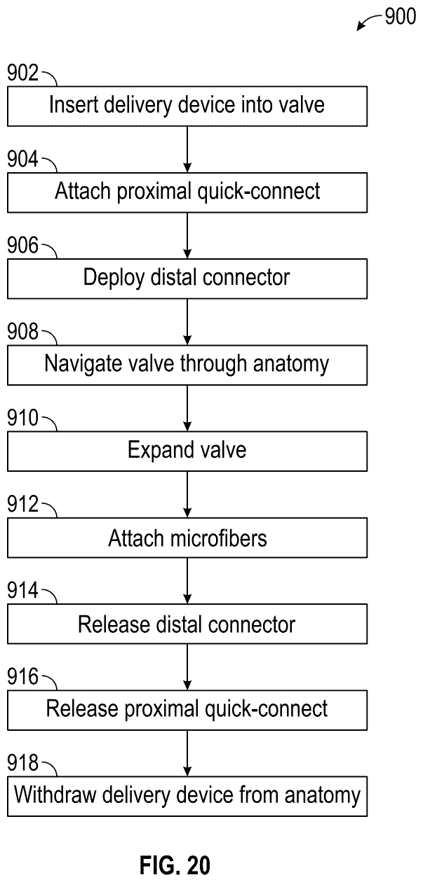

FIG. 20 is a block diagram illustrating methods of deploying and recovering bronchial valves of the present disclosure. Though discussed with reference to bronchial valve 100 and delivery system 400, method 900 can encompass the use of any valve and delivery system consistent with the valves and delivery systems described herein. Method 900 can additionally include fewer or greater operations other than operation 902 to operation 918. Additionally, in other examples, operation 902 through operation 918 can be performed in other sequences.

At operation 902, a delivery device can be coupled with a valve. In particular, a distal tip of a delivery catheter can be inserted into a lumen of a bronchial valve. For example, distal tip 422 can be inserted into lumen 112 of valve frame 114 of bronchial valve 100.

At operation 904, a proximal end of the valve can be attached to the delivery device. In particular, a proximal end of a bronchial valve can be connected to a twist lock or quick-connect coupler on the delivery catheter. For example, locking pegs 428 of lock ring 424 can be inserted into keyway 132 of coupler 110. Locking pegs 428 can be inserted axially into a first axially extending portion of keyway 132 and then attachment knob 410 can be rotated to cause locking pegs 428 to move down into a second circumferentially extending portion of keyway 132.

At operation 906, a distal end of the valve can be attached to the delivery device. In particular, a distal end of a bronchial valve can be connected to a deployable coupler on the delivery catheter. For example, locking balls 426 of distal tip 422 can be inserted into slots between fingers 130 on valve frame 114 of bronchial valve 100. Knob 450 of locking knob 406 can be actuated to allow locking balls 426 to be freely moveable in sockets 440 by advancing sliding block 430 distally. Once locking balls 426 are positioned between fingers 130, knob 450 can be actuated to move sliding block 430 proximally toward biasing block 434 to fix locking balls 426 into a radially extended position in engagement with valve frame 114.

At operation 908, the valve can be moved to a desired location using the delivery system. In particular, a bronchial valve can be navigated through anatomy to an anatomic duct. For example, bronchial valve 100 can be navigated through a mouth, esophagus and into a lung. Within the lung, bronchial valve 100 can be positioned within a bronchi, bronchioles or alveoli of the lung. Bronchial valve 100 can be delivered through a bronchoscope using imaging therefrom to position bronchial valve 100 in a desired position. Bronchial valve 100 can be positioned within a diseased or non-functioning bronchi, bronchioles or alveoli. The configuration of membrane 108 can allow bronchial valve 100 to freely slide axially against surfaces until microfibers 304 are activated.

At operation 910, the valve can be deployed by radially expanding the valve to contact walls of an anatomic duct. In particular, a bronchial valve can be radially expanded to contact a wall of the bronchi, bronchioles or alveoli. For example, bronchial valve 100 can be expanded to contact duct surface 504. Bronchial valve 100 can be expanded by rotating deployment knob 408. Deployment knob 408 can be rotated to turn screw 414 to axially displace inner tube 416. Inner tube 416 can be used to push or pull lock ring 424. In examples, deployment knob 408 can pull locking balls 426 closer to locking pegs 428. In an example, deployment knob 408 can push lock ring 424 distally to move locking pegs 428 closer to locking balls 426. As discussed herein, relative axial movement between locking pegs 428 and locking balls 426 can cause expansion and contraction of valve frame 114 of bronchial valve 100 for deployment and recovery, respectively. A user can operate deployment knob 408 to controllably expand valve frame 114 to contact duct surface 504, thereby customizing the deployed diameter of bronchial valve to match the anatomic shape of the specific patient. The user can utilize video feedback from a bronchoscope to visualize the relative position and size of bronchial valve 100 to anatomic passage 500.

At operation 912, the valve can be expanded to engage a microfiber array with the walls of the anatomic duct. In particular, a bronchial valve can be expanded to push mushroom shaped microfibers into the wall of the bronchi, bronchioles or alveoli. For example, heads 316 of microfibers 304 can be forced axially into duct surface 504 to cause face 318 to adhere thereto. Such adhesion of face 318 can be caused by temporarily overexpanding valve frame 114 to cause the desired engagement of microfibers 304. After microfibers 304 are adhered to duct surface 504, valve frame 114 can be contracted to the desired size, e.g., to block bronchi, bronchioles or alveoli without inducing significant stress or strain within duct surface 504. As valve frame 114 is contracted to the desired size, stems 314 of microfibers 304 can be stretched to accommodate differences between the outer diameter of bronchial valve 100 and anatomic passage 500.

At operation 914, a distal end of the valve can be detached from the delivery device. In particular, a distal end of a bronchial valve can be disconnected from a deployable coupler on the delivery catheter. For example, locking balls 426 of distal tip 422 can be withdrawn from slots between fingers 130 on valve frame 114 of bronchial valve 100. Knob 450 of locking knob 406 can be actuated to allow locking balls 426 to be freely moveable in sockets 440 by retracting sliding block 430 proximally from biasing block 434.

At operation 916, a proximal end of the valve can be detached from the delivery device. In particular, a proximal end of a bronchial valve can be disconnected from a quick-connect coupler on the delivery catheter. For example, locking pegs 428 of lock ring 424 can be withdrawn from keyway 132 of coupler 110. Attachment knob 410 can be rotated to cause locking pegs 428 to move upward out of the second circumferentially extending portion of keyway 132 and then locking pegs 428 can be withdrawn axially from the first axially extending portion of keyway 132.

At operation 918, the delivery device can withdrawn from the anatomy of the patient. In particular, a distal tip of a delivery catheter can be withdrawn from the lumen of the bronchial valve. For example, distal tip 422 can be withdrawn from lumen 112 of valve frame 114 of bronchial valve 100 to allow delivery system 400 to be withdrawn from the anatomy and a bronchoscope.

Whenever it is desired to remove bronchial valve 100 for the anatomy, operation 902 through operation 918 can be performed again to collapse bronchial valve 100 instead of expanding. In examples, operation 902 to operation 918 can be performed in reverse order to retrieve bronchial valve 100, though other sequences of operations can be used. A new delivery device can be used from the one used to perform insertion.

The present disclosure describes bronchial valve systems designed for the treatment of Chronic Obstructive Pulmonary Disease (COPD) and related pulmonary conditions. The benefits of the bronchial valves, delivery systems and associated methods are numerous.

Targeted Lung Volume Reduction: Bronchial valves of the present disclosure provides a non-surgical option for lung volume reduction, which can be important for patients with severe COPD in order to provide improved breathing. By obstructing air passageways to non-functioning lung sections, the bronchial valves of the present disclosure allow healthier parts of the lung to perform better, potentially improving the breathing of the patient and overall lung function.

Removability and Adjustability: A benefit of the bronchial valves of the present disclosure is that the valve can be removed and adjusted. Removability and adjustability allow for non-permanent placement, meaning the valve can be adjusted or removed if the patient's condition changes or if there are complications.

Enhanced Patient Comfort: The design of the bronchial valves of the present disclosure can help ensure that a membrane surrounding the valve engages fluish with an anatomic duct in a fully expanded or partially expanded state to reduce tissue strain and patient discomfort. This can also contribute to a better seal in irregularly shaped airways and enhances patient comfort by minimizing irritation.

Improved Treatment Efficacy: The ability of the bronchial valves of the present disclosure to expand to different sizes and its use of a one-way valve mechanism to block inflow while permitting outflow can significantly improve the efficacy of the treatment. This design can help in effectively reducing the lung volume while allowing trapped air and fluids to escape, which can alleviate symptoms of breathlessness.

Prevention of Migration: The use of microfibers on membranes used with the bronchial valves can help in attaching the device securely to the anatomic surfaces, preventing migration post-deployment. This can be important for maintaining the effectiveness of the treatment over time.

Minimally Invasive Procedure: The deployment of the bronchial valves of the present disclosure can use a catheter-based delivery system that is minimally invasive compared to surgical options. This can reduce the risk associated with surgeries and can lead to quicker recovery times. Furthermore, complicated loading procedures and loading devices need not be used to attach a bronchial valve of the present disclosure.

Catheter-Based Delivery: The bronchial valves can be delivered and deployed using a catheter, which is minimally invasive and allows for precise placement of the valve within the bronchial tubes.

Controlled Deployment: The deployment mechanisms of the present disclosure can include a handle with various user-friendly actuation features to deploy, expand, and release the valves. This allows for controlled and accurate placement, which is critical for the success of the treatment.

Adjustability During Deployment: The bronchial valves of the present disclosure can be repositioned or adjusted during the deployment process. This flexibility ensures that the valve can be optimally placed to achieve the best possible therapeutic outcome.

Safety Features: The deployment process includes several safety features, such as the ability to reattach the valve to the delivery system and return it to a collapsed state without causing tissue trauma. This is advantageous for ensuring patient safety during and after the procedure.

In summary, the bronchial valve system described in the document provides a significant advancement in the treatment of COPD and similar pulmonary conditions. The bronchial valve system offers a combination of effectiveness, safety, and patient comfort that makes it a valuable alternative to more invasive surgical procedures.

### EXAMPLES

Example 1 is a bronchial flow control device comprising: a tubular body comprising: a first end; a second end; and a plurality of struts extending between the first end and the second end; wherein the plurality of struts is configured to expand from a radially contracted shape to a radially expanded shape; a valve disposed proximate the second end; a membrane covering the plurality of struts; and a plurality of microfibers disposed about an exterior of the membrane.

In Example 2, the subject matter of Example 1 optionally includes wherein: the first end comprises a first coupler for attaching to a delivery device; and the second end comprises a second coupler for attaching to the delivery device.

In Example 3, the subject matter of Example 2 optionally includes wherein: the first coupler comprises a keyway slot; and the second coupler comprises a slot for a ball.

In Example 4, the subject matter of any one or more of Examples 1-3 optionally include wherein the valve comprises a one-way valve.

In Example 5, the subject matter of any one or more of Examples 1-4 optionally include wherein the valve comprises a ball valve or a flapper valve.

In Example 6, the subject matter of any one or more of Examples 1-5 optionally include wherein the membrane is disposed on an outside of the tubular body.

In Example 7, the subject matter of Example 6 optionally includes wherein the membrane is allowed to slide along an exterior surface of the tubular body.

In Example 8, the subject matter of any one or more of Examples 1-6 optionally include wherein the membrane is positioned between the first end and the second end of the tubular body.

In Example 9, the subject matter of any one or more of Examples 1-8 optionally include one or more shrink wrap bands to secure the membrane to the tubular body.

In Example 10, the subject matter of any one or more of Examples 1-9 optionally include wherein the plurality of microfibers includes mushroom-head shaped microfibers.

In Example 11, the subject matter of any one or more of Examples 1-10 optionally include wherein each of the plurality of microfibers has an elongate stem and a head attached to a distal end of the elongate stem.

In Example 12, the subject matter of any one or more of Examples 1-11 optionally include wherein the tubular body comprises a first ridge and a second ridge bounding ends of the membrane.

In Example 13, the subject matter of any one or more of Examples 1-12 optionally include wherein at least one of the plurality of struts is preconfigured to deform to the radially expanded shape.

In Example 14, the subject matter of any one or more of Examples 1-13 optionally include wherein the radially expanded shape comprises a bulbous shape.

In Example 15, the subject matter of any one or more of Examples 13-14 optionally include wherein at least one of the plurality of struts includes a middle segment that is positioned further radially outward than ends of the at least one of the plurality of struts.

In Example 16, the subject matter of any one or more of Examples 13-15 optionally include wherein at least one of the plurality of struts is circumferentially wider proximate to a center of the at least one of the plurality of struts compared to ends of the at least one of the plurality of struts.

In Example 17, the subject matter of any one or more of Examples 13-16 optionally include wherein each of the plurality of struts comprises: a proximal straight end; a proximal tapered portion; a middle segment; a distal tapered portion; and a distal straight end.

In Example 18, the subject matter of any one or more of Examples 1-17 optionally include wherein each of the plurality of struts comprises a strut having a rectilinear cross-sectional shape.

In Example 19, the subject matter of any one or more of Examples 1-18 optionally include wherein the first end of the valve and the second end of the valve have approximately equal diameters.

Example 20 is a method of deploying a bronchial valve in an airway, the method comprising: attaching the bronchial valve to a delivery catheter in an unexpanded state; inserting the delivery catheter with the bronchial valve into the airway; operating an actuator of the delivery catheter to radially expand the bronchial valve; pushing microfibers of the bronchial valve into an anatomic duct wall of the airway; detaching the delivery catheter from the bronchial valve; and withdrawing the delivery catheter from the airway.

In Example 21, the subject matter of Example 20 optionally includes attaching a proximal quick-connect of the delivery catheter to a proximal end of the bronchial valve; and attaching a distal coupler of the delivery catheter to a distal end of the bronchial valve.

In Example 22, the subject matter of Example 21 optionally includes operating an actuator of the delivery catheter to radially expand the bronchial valve comprises drawing a proximal end and a distal end of the bronchial valve together to push a portion of a valve frame of the bronchial valve radially outward.

In Example 23, the subject matter of Example 22 optionally includes wherein operating an actuator of the delivery catheter to radially expand the bronchial valve comprises mechanically deforming the valve frame of the bronchial valve.

In Example 24, the subject matter of any one or more of Examples 20-23 optionally include wherein pushing microfibers of the bronchial valve into an anatomic duct wall of the airway comprises pushing a mushroom head shaped microfiber into the anatomic duct wall.

Example 25 is a delivery system for a valve, the delivery system comprising: a handle; a first actuator connected to the handle to control a rotational position of a first coupler; a second actuator connected to the handle to control a radial position of a second coupler; and a third actuator to control an axial position between the first coupler and the second coupler.

In Example 26, the subject matter of Example 25 optionally includes an internal passage in the handle; an inner tube connected to the third actuator in the internal passage and extending out of the handle; a wire extending from the second actuator and extending from the internal passage out of the handle and into the inner tube; and an outer tube connected to the first actuator and extending from the handle around the inner tube; wherein operation of the first actuator can rotate the outer tube; wherein operation of the second actuator can lock a radial position of the second actuator; and wherein operation of the third actuator can push or pull the inner tube to move the second coupler axially relative to the first coupler.

In Example 27, the subject matter of Example 26 optionally includes wherein: the first coupler comprises quick-connect coupler comprising a plurality of pegs extending from a ring to engage keyway slots on a bronchial valve; and the second coupler comprises a plurality of locking balls that can be radially displaced to engage slots on the bronchial valve.

In Example 28, the subject matter of Example 27 optionally includes wherein: the first actuator comprises a knob located at a distal end of the handle; the second actuator comprises knob located at a proximal end of the handle; and the third actuator comprises a knob located on the handle between the first actuator and the second actuator.

Each of these non-limiting examples can stand on its own, or can be combined in various permutations or combinations with one or more of the other examples.

### Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F. R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A bronchial flow control device comprising:
a tubular body comprising:
a first end;
a second end; and
a plurality of struts extending between the first end and the second end;
wherein the plurality of struts is configured to expand from a radially contracted shape to a radially expanded shape;
a valve disposed proximate the second end;
a membrane covering the plurality of struts; and
a plurality of microfibers disposed about an exterior of the membrane.

2. The bronchial flow control device of claim 1, wherein:
the first end comprises a first coupler for attaching to a delivery device; and
the second end comprises a second coupler for attaching to the delivery device.

3. (canceled)

4. The bronchial flow control device of claim 1, wherein the valve comprises a one-way valve.

5. (canceled)

6. The bronchial flow control device of claim 1, wherein the membrane is disposed on an outside of the tubular body and the membrane is allowed to slide along an exterior surface of the tubular body.

7. (canceled)

8. The bronchial flow control device of claim 1, wherein the membrane is positioned between the first end and the second end of the tubular body.

9. The bronchial flow control device of claim 1, further comprising one or more shrink wrap bands to secure the membrane to the tubular body.

10. The bronchial flow control device of claim 1, wherein the plurality of microfibers includes mushroom-head shaped microfibers.

11. The bronchial flow control device of claim 1, wherein each of the plurality of microfibers has an elongate stem and a head attached to a distal end of the elongate stem.

12. The bronchial flow control device of claim 1, wherein the tubular body comprises a first ridge and a second ridge bounding ends of the membrane.

13. The bronchial flow control device of claim 1, wherein at least one of the plurality of struts is preconfigured to deform to the radially expanded shape.

14. The bronchial flow control device of claim 13, wherein the radially expanded shape comprises a bulbous shape.

15. The bronchial flow control device of claim 13, wherein at least one of the plurality of struts includes a middle segment that is positioned further radially outward than ends of the at least one of the plurality of struts.

16. The bronchial flow control device of claim 13 wherein at least one of the plurality of struts is circumferentially wider proximate to a center of the at least one of the plurality of struts compared to ends of the at least one of the plurality of struts.

17. The bronchial flow control device of claim 13, wherein each of the plurality of struts comprises:
a proximal straight end;
a proximal tapered portion;
a middle segment;
a distal tapered portion; and
a distal straight end.

18. (canceled)

19. (canceled)

20. A method of deploying a bronchial valve in an airway, the method comprising:
attaching the bronchial valve to a delivery catheter in an unexpanded state;
inserting the delivery catheter with the bronchial valve into the airway;
operating an actuator of the delivery catheter to radially expand the bronchial valve;
pushing microfibers of the bronchial valve into an anatomic duct wall of the airway;
detaching the delivery catheter from the bronchial valve; and
withdrawing the delivery catheter from the airway.

21. (canceled)

22. The method of claim 20, operating an actuator of the delivery catheter to radially expand the bronchial valve comprises drawing a proximal end and a distal end of the bronchial valve together to push a portion of a valve frame of the bronchial valve radially outward.

23. (canceled)

24. The method of claim 20, wherein pushing microfibers of the bronchial valve into an anatomic duct wall of the airway comprises pushing a mushroom head shaped microfiber into the anatomic duct wall.

25. A delivery system for a valve, the delivery system comprising:
a handle;
a first actuator connected to the handle to control a rotational position of a first coupler;
a second actuator connected to the handle to control a radial position of a second coupler; and
a third actuator to control an axial position between the first coupler and the second coupler.

26. The delivery system of claim 25, further comprising:
an internal passage in the handle;
an inner tube connected to the third actuator in the internal passage and extending out of the handle;
a wire extending from the second actuator and extending from the internal passage out of the handle and into the inner tube; and
an outer tube connected to the first actuator and extending from the handle around the inner tube;
wherein operation of the first actuator can rotate the outer tube;
wherein operation of the second actuator can lock a radial position of the second actuator; and
wherein operation of the third actuator can push or pull the inner tube to move the second coupler axially relative to the first coupler.

27. The delivery system of claim 26, wherein:
the first coupler comprises quick-connect coupler comprising a plurality of pegs extending from a ring to engage keyway slots on a bronchial valve; and
the second coupler comprises a plurality of locking balls that can be radially displaced to engage slots on the bronchial valve;
the first actuator comprises a knob located at a distal end of the handle;
the second actuator comprises knob located at a proximal end of the handle; and
the third actuator comprises a knob located on the handle between the first actuator and the second actuator.

28. (canceled)
